(19) United States
(12) Patent Application Publication
(10) Pub. No.: US 2024/0335620 A1
(43) Pub. Date: Oct. 10, 2024

(54) A cassette sub-assembly for a cassette unit

(71) Applicant: SHL Medical AG, Zug (CH)

(72) Inventors: Ming-Ting Yin, Taoyuan City (TW); Oscar Alexandersson, Nacka Strand (SE); Johan Zander, Nacka Strand (SE)

(21) Appl. No.: 18/716,332

(22) PCT Filed: Nov. 22, 2022
(86) PCT No.: PCT/EP2022/082689

§ 371 Date: Jun. 4, 2024

Related U.S. Application Data
(60) Provisional application No. 63/418,895, filed on Oct. 24, 2022.

(30) Foreign Application Priority Data
Dec. 6, 2021 (EP) 21212495.2

(51) Int. Cl. A61M 5/32

(52) U.S. Cl. CPC A61M 5/3245

(57) ABSTRACT

The present disclosure provides a cassette sub-assembly for a cassette unit configured to be detachably connected to a drive unit to form a medicament delivery device. The cassette sub-assembly includes a housing having a proximal housing end and a distal housing end, and a delivery member cover protruding from the proximal housing end and configured to be moved linearly relative to the housing. The cassette sub-assembly further includes a lock member arranged axially fixed relative to the housing, the lock member being configured to be rotated relative to the delivery member cover from a first position to a second position.

### CROSS-REFERENCE TO RELATED APPLICATIONS

The present application is a U.S. National Phase Application pursuant to 35 U.S.C. § 371 of International Application No. PCT/EP2022/082689 filed Nov. 22, 2022, which claims priority to (i) European Patent Application No. 21212495.2 filed Dec. 6, 2021, and (ii) U.S. Provisional Application No. 63/418,895 filed Oct. 24, 2022. The entire disclosure contents of these applications are herewith incorporated by reference into the present application.

### TECHNICAL FIELD

The present disclosure generally relates to medicament delivery devices.

### BACKGROUND

Some reusable medicament delivery devices may have a hybrid design, comprising a disposable first unit for example holding a syringe or a medicament cartridge provided with a needle, and a reusable second unit for example comprising a power pack. The power pack may include a spring-loaded, motor-driven, or gas-driven plunger rod.

The first unit may comprise a linearly movable needle cover, which covers the needle when the needle cover is in a first position. The needle cover may be moved linearly to a second position in which the needle is exposed. Due to this construction, there is a risk of accidental needle sticks if the needle cover is unintendedly moved to the second position.

### SUMMARY

There is hence according to a first aspect of the present disclosure provided a cassette sub-assembly for a cassette unit configured to be detachably connected to a drive unit to form a medicament delivery device, the cassette sub-assembly comprising: a housing having a proximal housing end and a distal housing end, a delivery member cover protruding from the proximal housing end and configured to be moved linearly relative to the housing, a lock member arranged axially fixed relative to the housing, the lock member being configured to be rotated relative to the delivery member cover from a first position to a second position, wherein the lock member has a proximally facing radial surface and the delivery member cover has a distally facing radial surface, wherein the proximally facing radial surface is axially aligned with and facing the distally facing radial surface when the lock member is in the first position and wherein the proximally facing radial surface is circumferentially offset relative to the distally facing radial surface when the lock member is in the second position.

The delivery member cover is prevented from moving from an extended position to a retracted position relative to the housing when the lock member is in the first position, i.e., when the proximally facing radial surface is axially aligned with and facing the distally facing radial surface.

The delivery member cover is allowed to move from an extended position to a retracted position relative to the housing when the lock member is in the second position, i.e., when the proximally facing radial surface is circumferentially offset relative to the distally facing radial surface.

Linear movement of the delivery member cover is controlled by the rotational position of the lock member. Unintentional linear movement of the delivery member, e.g., to expose a delivery member such as a needle, can thus be prevented by the lock member.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment the lock member is arranged radially outside of the delivery member cover.

According to one embodiment the lock member is annular, is a circlip or has a half-circular shape.

According to one embodiment the delivery member cover has an axially extending rib, wherein a distal end face of the rib forms the distally facing radial surface, and the lock member has a ledge structure forming the proximally facing radial surface and a distally extending axial passageway next to the ledge structure in a circumferential direction, wherein the axial passageway is configured to receive the rib when the lock member is in the second position.

According to one embodiment the lock member has an axially extending rib, wherein a proximal end face of the rib forms the proximally facing radial surface, and the delivery member cover has a ledge structure forming the distally facing radial surface and a proximally extending axial passageway next to the ledge structure in a circumferential direction, wherein the axial passageway is configured to receive the rib when the lock member is in the second position.

According to one embodiment the ledge structure comprises a radial ledge surface and a protrusion extending axially from the radial ledge surface, wherein the protrusion is arranged between the radial ledge surface and the axial passageway in the circumferential direction of the lock member. Rotation of the lock member to the second position when the delivery member cover is pushed axially towards the retracted position may thereby be prevented.

According to one embodiment the delivery member cover is rotationally locked relative to the housing.

According to one embodiment the outer surface of the delivery member cover has a perimeter that has a non-circular cross-sectional shape and the housing has an inner surface with a cross-sectional shape that corresponds to the non-circular cross-sectional shape, the cross-sections being taken through a longitudinal axis of the cassette sub-assembly.

According to one embodiment the delivery member cover has an outer surface provided with a first structure and the housing has a second structure, the first structure being configured to engage with the second structure to prevent relative rotation between the delivery member cover and the housing.

According to one embodiment the first structure is an axial rib and the second structure is an axial channel, or wherein the first structure is an axial channel, and the second structure is an axial rib.

One embodiment comprises a fastening structure configured to engage with a counter fastening structure of the drive unit.

According to one embodiment the fastening structure forms one part of a bayonet connection or forms one part of a screw connection.

According to one example, the fastening structure is arranged on an outer surface of the housing.

Alternatively, the cassette sub-assembly may comprise a rear member configured to be axially and rotationally fixed relative to the housing, wherein the fastening structure is arranged on an outer surface of the rear member.

According to one example, the lock member has an outer surface provided with a first helical track or first rib. The drive unit may in this example comprises a second rib configured to run in the first helical track, or a second helical track configured to receive the first rib. The cassette unit comprising the cassette sub-assembly may in this case be inserted linearly into the drive unit, without rotation, and the first helical track or first rib acts as part of a cam structure causing rotation of the lock member from the first position to the second position. The cassette unit comprising the cassette sub-assembly may in this case for example comprise a snap fit structure configured to engage with the drive unit by means of a snap fit connection.

One embodiment comprises a medicament container holder arranged in the housing and configured to be moved from a distal position relative to the housing to a proximal position by the drive unit when the cassette sub-assembly is connected to the drive unit.

According to one embodiment the medicament container holder has an outer surface provided with a radially outwards extending bevelled surface and the housing has an inner surface provided with a radially inwards extending bevelled surface arranged proximally relative to the radially outwards extending bevelled surface and facing the radially outwards extending bevelled surface when the medicament container holder is in the distal position.

The radially outwards extending bevelled surface is configured to move past the radially inwards extending bevelled surface when the medicament container holder is subjected to a proximally directed axial force that is greater than a threshold value.

There is according to a second aspect provided a cassette unit for a medicament delivery device comprising the cassette sub-assembly of the first aspect.

There is according to a third aspect provided a medicament delivery device comprising the cassette unit of the second aspect and a drive unit configured to be detachably connected to the cassette unit.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an example of a cassette unit for a medicament delivery device;

FIG. 2 is an exploded view of the cassette unit in FIG. 1;

FIG. 3 depicts a perspective view of a delivery member cover and a lock member;

FIG. 4 is a perspective view of a lock member and a rear member;

FIGS. 5-6 show longitudinal sections of the cassette unit with the medicament container holder in a distal position and in a proximal position, respectively;

FIG. 7 shows a perspective view from the front of the cassette unit when the lock member is in the first position;

FIGS. 8-9 show perspective views of the cassette unit when the lock member is in the second position;

FIG. 10 is a perspective view of the cassette unit with the housing, the lock member and the rear member removed, and the delivery member cover shown in transparent after medicament delivery;

FIG. 11 is a perspective view of another example of the cassette sub-assembly of the invention, the cassette sub-assembly comprises the housing, the lock member, the rear member, and a cap;

FIG. 12 is an exploded view of the cassette unit in FIG. 11;

FIG. 13 is a perspective view of FIG. 11 that only the delivery member cover and the lock member are shown;

FIG. 14 is a cross-section view of another example of the cassette sub-assembly of the invention, the cassette sub-assembly comprises the housing, the lock member and the rear member;

FIG. 15 is a cross-section view of the cassette sub-assembly as shown in FIG. 14 with the housing, the lock member and the rear member.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

The present disclosure relates to a cassette sub-assembly for a cassette unit. The cassette unit is for a medicament delivery device.

The cassette sub-assembly comprises a housing, a delivery member cover that is linearly movable in the housing, and a lock member. The lock member is configured to be rotated relative to the delivery member cover from a first position to a second position.

In the first embodiment, the lock member has a proximally facing radial surface and the delivery member cover has a distally facing radial surface, which are axially aligned and facing each other when the lock member is in the first position. The lock member in the first position thus prevents movement in the distal direction of the delivery member cover.

In the second embodiment, the lock member has a distally facing radial surface and the delivery member cover has a proximally facing radial surface, which are axially aligned and facing each other when the lock member is in the first position. The lock member in the first position thus prevents movement in the proximal direction of the delivery member cover.

The proximally facing radial surface is arranged offset from the distally facing radial surface in the circumferential direction when the lock member is in the second position. The delivery member cover is thus free to move linearly when the lock member is in the second position. In particularly, in the first embodiment, the delivery member cover is free to move linearly in the distal direction when the lock member is in the second position; and the in second embodiment, the delivery member cover is free to move linearly in the proximal direction when the lock member is in the second position.

The cassette unit and a drive unit configured to be mechanically detachably connected to the cassette unit form a medicament delivery device.

The medicament delivery device may for example be an autoinjector or a manual injector.

The cassette unit may form a front, or proximal part, and the drive unit may form a rear, or distal part, of the medicament delivery device.

The cassette unit may be disposable. The drive unit may be reusable.

The cassette unit is configured to hold a medicament container. The drive unit is configured to empty the content of the medicament container by mechanical means such as a spring-loaded plunger rod, a motor-driven plunger rod, or a gas-driven plunger rod.

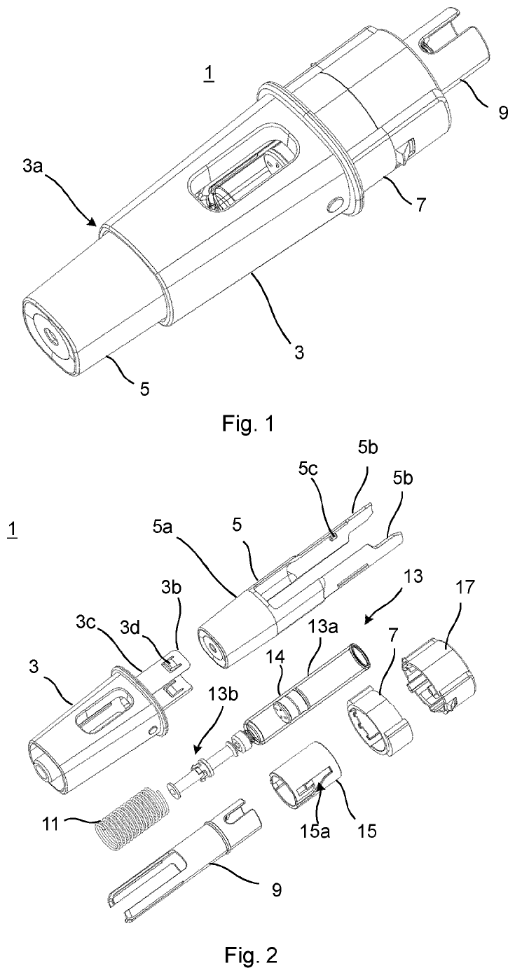

Examples of a cassette sub-assembly will now be described with reference to FIGS. 1-10 in the context of a main exemplary cassette unit.

FIG. 1 shows a perspective view of an example of a cassette unit 1 for a medicament delivery device. FIG. 11 shows a perspective view of another example of a cassette unit 1 for a medicament delivery device.

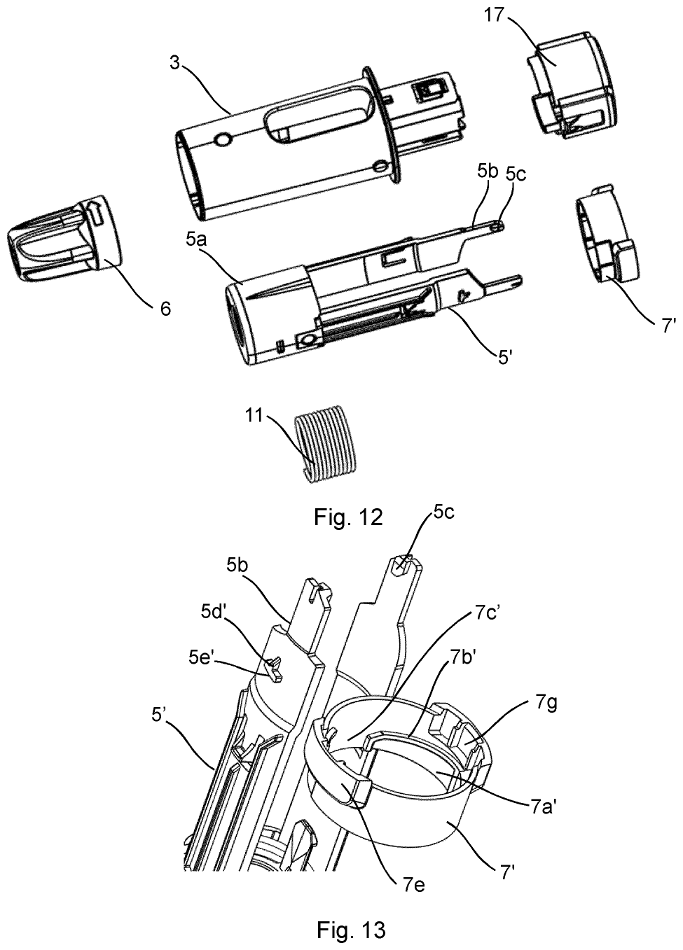

The cassette unit 1; 1′ comprises a housing 3. The housing 3 has a proximal housing end 3a and a distal housing end 3b, shown in FIG. 2 and FIG. 12. The housing 3 has a proximal opening arranged at the proximal housing end 3a.

The cassette unit 1; 1′ comprises a delivery member cover 5; 5′; 5″. The delivery member cover 5; 5′; 5″ is partly arranged in the housing 3. The delivery member cover 5; 5′; 5″ protrudes in the proximal direction from the proximal opening of the housing 3.

In one example, the delivery member cover 5; 5′ is configured to be rotationally locked relative to the housing 3. The delivery member cover 5; 5′ may for example have an outer surface that has perimeter with a non-circular cross-sectional shape, the section being taken perpendicular to a longitudinal axis of the delivery member cover 5; 5′. The housing 3 may have a corresponding non-circular cross-sectional shape. The proximal opening may in particular have a corresponding non-circular cross-sectional shape.

Alternatively, the delivery member cover 5″ is rotatable relative to the housing 3 within certain rotational angles around the longitudinal axis depends on the design.

Alternatively, the delivery member cover 5; 5′; 5″ may be provided with a first structure and the housing may be provided with a second structure configured to engage with each other such that the delivery member cover 5; 5′ is completely prevented from rotating relative to the housing or the delivery member cover 5″ is prevented from rotating relative to the housing beyond certain rotational angles around the longitudinal axis. One of these structures may for example be a rib and the other one may be an axial channel configured to engage with the rib.

The delivery member cover 5; 5′; 5″ is movable from an extended position to a retracted position relative to the housing 3. The delivery member cover 5; 5′; 5″ is biased towards the extended position.

The delivery member cover 5; 5′; 5″ may be arranged to return to the extended position from the retracted position. The delivery member cover 5; 5′; 5″ is then according to one example locked out and cannot be moved to the retracted position again.

The cassette unit 1 comprises a lock member 7; 7′. The lock member 7; 7′ may for example be annular, be a circlip or have a half-circular shape.

The lock member 7; 7′ is axially fixed relative to the housing 3.

The lock member 7; 7′ is configured to be rotated relative to the delivery member cover 5; 5′; 5″. The lock member 7; 7′ is configured to be rotated relative to the housing 3.

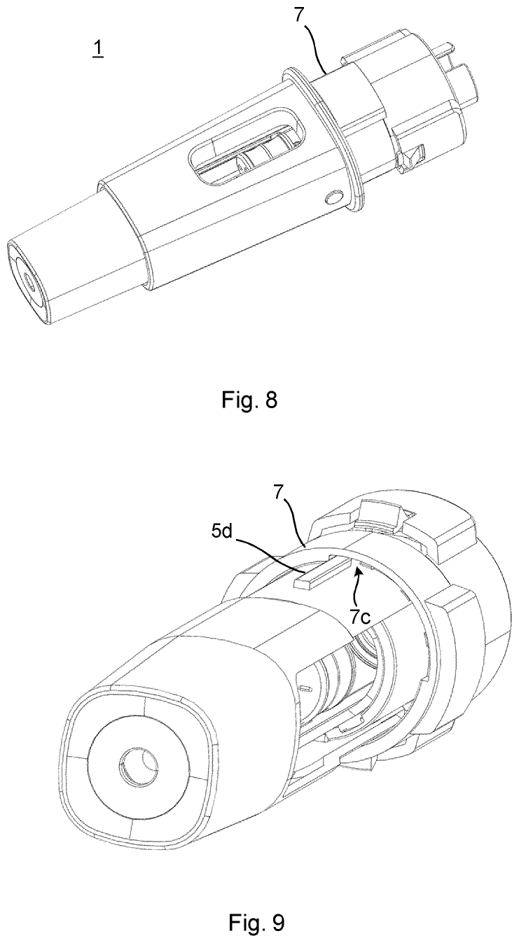

The lock member 7; 7′ is configured to be rotated from a first position, shown in FIG. 1, FIG. 11 and FIGS. 14-15, to a second position shown in FIG. 8. In the first position, an axial movement of the delivery member cover 5; 5′; 5″ is blocked by the lock member 7; 7′. In the second position, the axial movement of the delivery member cover 5; 5′; 5″ is no longer blocked by the lock member 7; 7′. In the first embodiment, in the first position, the lock member 7 blocks the delivery member cover 5 from moving to the retracted position. In the second position, the delivery member cover 5 is allowed to move from the extended position to the retracted position. The lock member 7 does in the second position no longer block the delivery member cover 5 from moving to the retracted position.

In the second embodiment, as shown in FIG. 13, in the first position, the lock member 7′ blocks the delivery member cover 5′ from moving to the extended position. In the second position, the delivery member cover 5′ is allowed to move to the extended position. The lock member 7′ does in the second position no longer block the delivery member cover 5′ from moving to the extended position.

It should be noted that the lock member can also prevent the delivery member cover from axially moving in both the distal direction and the proximal direction relative to the housing. For example, a circumferential groove can be arranged in a wall of the lock member. The circumferential groove defines the above-mentioned proximally facing radial surface and the distally facing radial surface of the lock member. The delivery member cover can comprise a protrusion positioned within the circumferential groove of the lock member when the lock member is in the first position. The groove may comprise a cut-out at one end such that the protrusion of the delivery member cover can move out of the groove via the cut-out. In this example, when the lock member is in the second position, the protrusion of the delivery member cover is away from the groove such that the axial movement of the delivery member cover is no longer blocked by the lock member. The housing 3, the delivery member cover 5; 5′, and the lock member 7; 7′ form or form part of a cassette sub-assembly.

In another example, the cassette unit 1 optionally comprises a medicament container holder 9; 9′ extending axially inside the housing 3. The medicament container holder 9; 9′ is configured to hold or accommodate a medicament container such as a syringe.

In one example, initially, when the delivery member cover 5 is in the extended position, and the lock member 7 is in the first position, the medicament container holder 9 is held in a distal position. The medicament container holder 9 is configured to be moved axially in the proximal direction, to a proximal position when the medicament container is subjected to a proximally directed force greater than a threshold value. This is typically done when the cassette unit 1 is being mechanically connected to a drive unit.

Alternatively, the medicament container holder 9′ can be fixed to housing 3, namely, the container holder is axially immovable relative to the housing 3.

FIG. 2 and FIG. 12 show exploded views of the cassette unit 1 in the first embodiment and in the second embodiment respectively. The exemplified housing 3 comprises distally extending axial housing legs 3c provided with radially flexible tabs 3d that extend radially outwards.

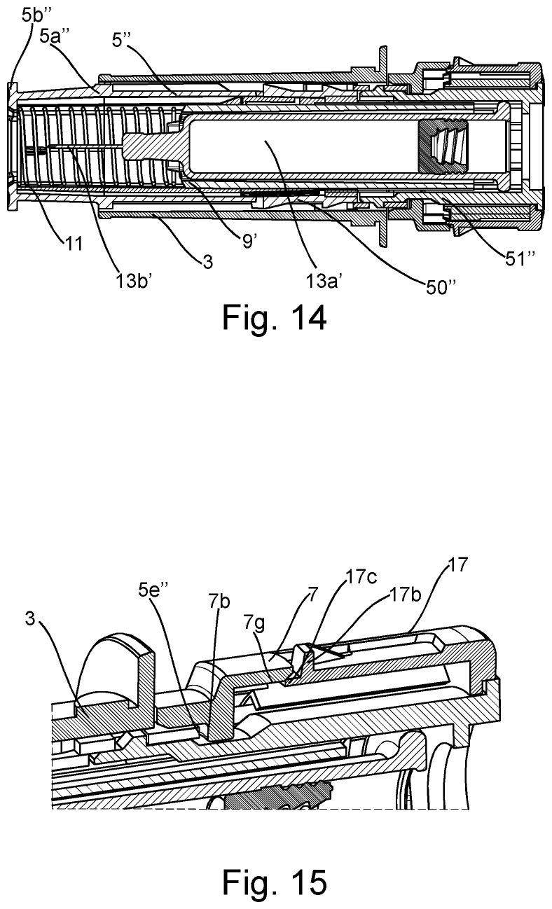

The delivery member cover 5; 5′; 5″ has a proximal circumferentially closed portion 5a; 5a″. The proximal circumferentially closed portion 5a may have a non-circular cross-sectional shape in a section perpendicular to the longitudinal axis of the delivery member cover 5; 5′. In another example, the delivery member cover 5″ comprises a flange 5b″ extending from the proximal circumferentially closed portion 5a″ in the direction transverse to the longitudinal axis, as shown in FIG. 14. In a preferred example, the flange 5b″ has a diameter greater than a diameter of the proximal opening of the housing 3 such that the flange 5b″ can limit the movement distance of the delivery member cover 5″ in the distal direction relative to the housing 3. Furthermore, in one example, the delivery member cover 5 is formed as one single component, as shown in FIG. 2. Alternatively, the delivery member cover 5″ is formed by multiple components that attached to one another. For example, the delivery member cover 5″ is formed by a proximal part 50″ and a distal part 51″ attached to one another, e.g., snap-fit, as shown in FIG. 14.

The delivery member cover 5; 5′; 5″ has axially extending legs 5b extending distally from the proximal circumferentially closed portion 5a. Each axially extending legs 5b is provided with a respective radially inwards extending protrusion 5c, such as a pin.

The cassette sub-assembly comprises a resilient member 11. The resilient member 11 is configured to bias the delivery member cover 5; 5′; 5″ towards the extended position. The resilient member 11 may be a spring, such as a coil spring, as shown in FIG. 14.

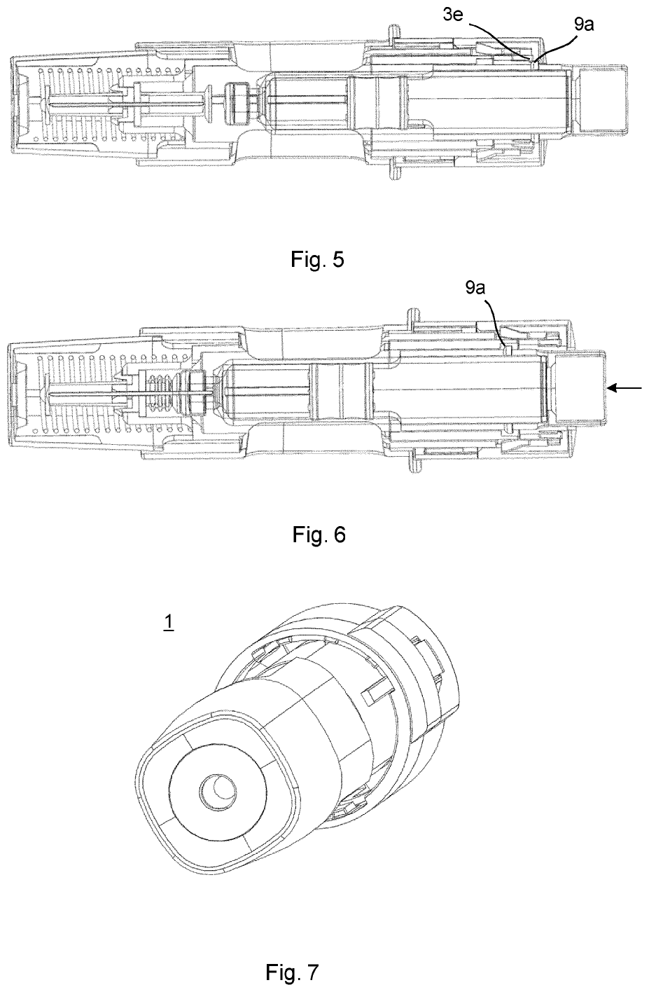

A medicament container 13, such as a syringe, may be arranged in the medicament container holder 9; 9′. The medicament container 13 may comprise a liquid container 13a including a stopper 14, and delivery member such as a double-edged needle 13b. The double-edged needle 13b may be covered with a proximal flexible tube and a distal flexible tube. In one example as shown in FIGS. 5-6, the double-edged needle 13b are configured to be attached to the liquid container 13a when the medicament container holder 9 moves in the proximal direction relative to the double-edged needle 13b. Alternatively, the double-edged needle 13b′ is integrated to the proximal end of the liquid container 13a′, as shown in FIG. 14.

Furthermore, in another example, the exemplified cassette unit 1 comprises a rotator 15. The rotator 15 is arranged around the medicament container holder 9. The delivery member cover 5 is in the example arranged radially outside of the rotator 15. The rotator 15 has a guide structure 15a including a helical track and an axial track arranged adjacent to the helical track in the circumferential direction. In use, each radially inwards extending protrusion 5c of the delivery member cover 5 is arranged to cooperate with a respective guide structure 15a. When the delivery member cover 5 is moved from the extended position to the retracted position, the radially inwards extending protrusions 5c run in a respective helical track. This causes rotation of the rotator 15, because the delivery member cover 5 is rotationally locked relative to the housing 3. When the delivery member cover 5 is returned to the extended position, the radially inwards extending protrusions 5c run back towards a proximal rotator end in a respective one of the axial tracks. The axial tracks are provided with a respective flexible element which the radially inwards extending protrusions 5c can pass when the delivery member cover 5 is moved in the proximal direction. The flexible elements will however prevent the delivery member cover 15 from moving back to the retracted position when the delivery member cover 5 has returned to the extended position. The delivery member cover 5 is thus locked out in the extended position after use.

In a preferred example, the cassette sub-assembly comprises a rear member 17. The rear member 17 may be annular, be a circlip or have a half-circular shape. The rear member 17 is arranged around the distally extending axial housing legs 3c. The radially flexible tabs 3d of the housing 3 are configured to engage with a snap fit connection with the rear member 17. The rear member 17 and the housing 3 are thus axially and rotationally fixed relative to each other. In this example, the medicament container can be inserted into the housing 3 from the distal end of the housing either during a manufacture process of the cassette unit or by a distributor or a user of the cassette unit, e.g., a patients, pharmacist, and other caregiver. It should be noted that the rear member can be an independent component attached to the housing, as the example mentioned above. Alternatively, the rear member 17 is integral to the housing, dependent on the selection of the manufacture process.

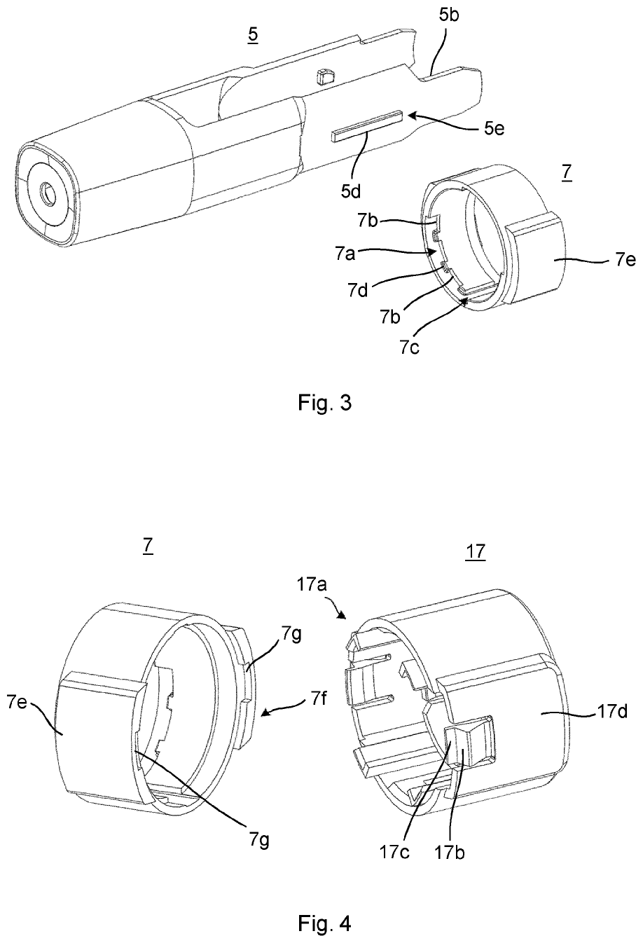

FIG. 3 shows a perspective view of the delivery member cover 5; 5″ of the first embodiment and the lock member 7 of the first embodiment. According to the example, the lock member 7 is configured to be arranged radially outside the delivery member cover 5; 5″.

In one example, the delivery member cover 5 has one or more axially extending rib 5d. The one or more axially extending rib 5d is provided on an outer surface of the delivery member cover 5. In the present example, each axially extending rib 5b is arranged on an outer surface of an axially extending leg 5b.

The axially extending rib 5d has a distal end face that forms a distally facing radial surface 5e.

The lock member 7 comprises a ledge structure 7a. The ledge structure 7a has one or more radial ledge surfaces, each being a proximally facing radial surface 7b. Each proximally facing radial surface 7b extends radially inwards from an inner surface of the lock member 7.

The lock member 7 has a distally extending axial passageway 7c arranged next to the ledge structure 7a in a circumferential direction of the lock member 7. The axial passageway 7c extends distally relative to the ledge structure 7a.

The ledge structure 7a comprises one or more protrusions, or bumps, 7d extending axially from a proximally facing radial surface 7b. Each protrusion 7d extends from a proximally facing radial surface 7b in the proximal direction. Each protrusion 7d is arranged between a proximally facing radial surface 7b and the axial passageway 7c in the circumferential direction of the lock member 7. The one or more protrusions 7d are configured to prevent the lock member 7 to rotate relative to the delivery member cover 5 if the delivery member cover is pushed in the distal direction. The circumferential distance between adjacent protrusions 7d is large enough to receive the axially extending rib 5d between them.

When the lock member 7 is in the first position, the axially extending rib 5d is arranged axially aligned with a proximally facing radial surface 7b or protrusion 7d. The distally facing surface 5e is thus axially aligned with and faces a proximally facing radial surface 7b or a proximally directed radial surface of a protrusion. The delivery member cover 5 is therefore prevented from moving from the extended position to the retracted position.

When the lock member 7 is in the second position, the axially extending rib 5d is arranged axially aligned with the axial passageway 7c. The axial passageway 7c has a circumferential width dimension configured to receive the axially extending rib 5d. The delivery member cover 5 may thus in this case be moved to the retracted position, with the axial passageway 7c receiving the axially extending rib 5d.

As an alternative to the above described design, the inner surface of the lock member could be provided with the axially extending rib and the outer surface of the delivery member cover could be provided with the ledge structure and the axial passageway.

Alternatively, the distally facing surface 5e″ is defined by the distal end of the delivery member guard 5″, as shown in FIG. 15. In this example, the lock member 7 does not be arranged with the protrusions 7d. In this example, the ledge structure 7a of the lock member 7 comprises the proximally facing radial surface 7b and the axial passageway 7c.

As another alternative, the lock member could be arranged radially inside the delivery member cover. In this case, the outer surface of the lock member may be provided with the ledge structure and the axial passageway, and the inner surface of the delivery member may be provided with the axially extending rib. A variation would be to provide the outer surface of the lock member with the axially extending rib and the inner surface of the delivery member cover with the ledge structure and the axial passageway.

The lock member 7 shown in FIG. 3 has an outer surface provided with lock member radially outwards extending structures 7e configured to engage with corresponding recesses in a drive unit.

Alternatively, FIG. 13 shows a perspective view of the delivery member cover 5′ of the second embodiment and the lock member 7′ of the second embodiment. According to the example, the lock member 7′ is configured to be arranged radially outside the delivery member cover 5′.

The delivery member cover 5′ has one or more protrusion 5d′. The one or more protrusion 5d′ is provided on an outer surface of the delivery member cover 5′. In the present example, each protrusion 5b′ is arranged on an outer surface of an axially extending leg 5b.

The protrusion 5d′ has a distal end face that forms a proximally facing radial surface 5e.

The lock member 7′ comprises a ledge structure 7a′. The ledge structure 7a′ has one or more radial ledge surfaces, each being a distally facing radial surface 7b′. Each proximally facing radial surface 7b′ extends radially inwards from an inner surface of the lock member 7′.

The lock member 7 has a distally extending axial passageway 7c arranged next to the ledge structure 7a′ in a circumferential direction of the lock member 7. The axial passageway 7c′ extends proximally relative to the ledge structure 7a′. In one preferred example, the the ledge structure 7a′ extends helically. In this example, the proximally facing radial surface 7b′ is a helical surface, as shown in FIG. 13.

When the lock member 7′ is in the first position, the protrusion 5d′ is arranged axially aligned with the distally facing radial surface 7b′. The proximally facing surface 5e′ is thus axially aligned with and faces the distally facing radial surface 7b′. The delivery member cover 5′ is therefore prevented from moving to the extended position.

When the lock member 7′ is in the second position, the protrusion 5d′ is arranged axially aligned with the axial passageway 7c′. The axial passageway 7c′ has a circumferential width dimension configured to receive the protrusion 5d′. The delivery member cover 5′ may thus in this case be moved to the extended position, e.g., by the resilient member 11 or bring manually pulled by the user, with the axial passageway 7c′ receiving the protrusion 5d′.

As an alternative to the above-described design, the inner surface of the lock member could be provided with the protrusion and the outer surface of the delivery member cover could be provided with the ledge structure and the axial passageway.

As another alternative, the lock member could be arranged radially inside the delivery member cover. In this case, the outer surface of the lock member may be provided with the ledge structure and the axial passageway, and the inner surface of the delivery member may be provided with the protrusion. A variation would be to provide the outer surface of the lock member with the protrusion and the inner surface of the delivery member cover with the ledge structure and the axial passageway.

Similar to the first embodiment, the lock member 7′ shown in FIG. 13 has an outer surface provided with lock member radially outwards extending structures 7e configured to engage with corresponding recesses in a drive unit.

FIG. 4 shows the lock member 7 and the rear member 17. The rear member 17 is arranged coaxially with the lock member 7. The rear member 17 is arranged distally relative to the lock member 7. It should be noted that the interaction between the lock member and the rear member will be explained in detail below with the first embodiment as an example. However, as the rear member 17 is configured to provide a safety mechanism to releasably lock the lock member from rotating from the first position to the second position before the cassette unit is connected to the drive unit, the rear member 17 can be used with both the first embodiment and the second embodiment as mentioned above.

The lock member 7 has a distal lock member end 7f provided with axial recesses 7g (as shown in FIG. 4 and FIG. 13). The rear member 17 has a proximal rear member end 17a provided with radially flexible structures 17b. The radially flexible structures 17b may for example be wedge-shaped, with an increasing radial dimension in a direction towards the proximal rear member end 17a. Each radially flexible structure 17b may be provided with an axially extending end tab 17c configured to be received by a respective axial recess 7g of the lock member 7. The lock member 7 is thereby rotationally fixed in the first position to the rear member 17. The rear member 17 is rotationally and axially fixed to the housing 3. The radially flexible structures 17b are pushed radially inwards by inner walls of an opening in the drive unit when the cassette unit 1 is inserted into and connected to the drive unit. This causes the axially extending tabs 17c to flex radially inwards and disengage from the respective axial recess 7g. The lock member 7 is thus set free to rotate from the first position to the second position.

The rear member 17 has fastening structures 17d configured to engage with a counter fastening structure of the drive unit. The fastening structures 17d are in the present example rear member radially outwards extending structures that are axially aligned with the lock member radially outwards extending structures 7e when the lock member 7 is in the first position. The rear member radially outwards extending structures form one part of a bayonet connection. The other part of the bayonet connection is provided in the drive unit.

When the cassette unit 1 is connected to the drive unit, the distal end of the cassette unit 1, including the lock member 7 and the rear member 17, is inserted into a proximal opening of the drive unit. The proximal opening of the drive unit has an inner surface provided with radial recesses configured to receive and guide a respective one of the fastening structures 17d and the lock member radially outwards extending structures 7e when the cassette unit 1 is inserted into the proximal opening of the drive unit. The radial recesses have an axial length corresponding to the axial length of the lock member radially outwards extending structures 7e, so that when the cassette unit 1 has been inserted into the drive unit, the fastening structures 17d are arranged axially beyond the axial recesses in the distal direction. The cassette unit 1 may be rotated relative to the drive unit when in this position. Since the lock member radially outwards extending structures 7e are arranged in the radial recesses, the lock member radially outwards extending structures 7e will maintain a fixed position relative to the drive unit, and the lock member 7 has been released, the rear member 17 and the housing 3 will be rotated relative to the drive unit and the lock member 7. The cassette unit 1 will thereby be locked with a bayonet connection to the drive unit. The lock member 7 is during the rotation between the cassette unit 1 and the drive unit set in the second position.

Alternatively, the lock member could be provided with the radially flexible structures and the axially extending end tabs at a proximal lock member end. The housing, in particular a radially outwards extending flange arranged directly proximally relative to the lock member, may be provided with axial recesses configured to receive the axially extending end tabs when the lock member is in the first position.

The fastening structure could instead of forming one part of a bayonet connection for example form one part of a screw connection.

Turning back to the first embodiment, as mentioned above, in one example where the ledge structure 7a of the lock member 7 comprises the protrusion 7d, the protrusion 7d of the lock member 7 is configured to prevent the rotation of the lock member 7 relative to the delivery member cover 5. Thus, when the user inserts the cassette unit 1 into the drive unit and pushes the delivery member cover 5 in the distal direction relative to the housing 3, but not yet rotates the cassette unit 1 relative to the drive unit and then the user pushes the delivery member cover 5, the axially extending rib 5d will radially align with the protrusion 7d, thereby, the rotation of the lock member 7 relative to the delivery member cover 5 is blocked. The delivery member cover 5 is rotationally fixed to the housing as mentioned above, thereby, when the axially extending rib 5d is radially align with the protrusion 7d, the lock member 7 will be prevented from rotating relative to the delivery member cover 5. As mentioned above, the lock member 7 will be prevented from rotating relative to the drive unit by the radial recess of the drive unit, as the result, the user will not be able to rotate the cassette unit relative to the drive unit. The user thereby gets an indication (from not able to attach the cassette unit to the drive unit) and is indicated that the delivery member cover 5 should not be pressed before the cassette is properly attached to the drive unit.

FIG. 5 shows a longitudinal section of the cassette unit 1 in an initial default state. The medicament container holder 9 is arranged in the distal position relative to the housing 3. In this case, a septum sealing the liquid container 13a is intact.

The medicament container holder 9 has an outer surface provided with a radially outwards extending bevelled surface 9a. The housing 3 has an inner surface provided with a radially inwards extending bevelled surface 3e. The radially inwards extending bevelled surface 3e is arranged proximally relative to the radially outwards extending bevelled surface 9a and facing the radially outwards extending bevelled surface 9a when the medicament container holder 9 is in the distal position.

The radially outwards extending bevelled surface 9a is configured to move past the radially inwards extending bevelled surface 3e when the medicament container holder 9 is subjected to a proximally directed axial force that is greater than a threshold value. The configuration of the bevelled surfaces 9a and 3e thus prevent proximal movement of the medicament container holder 9 until a large enough proximally directed axial force is acting on a proximal end face of the medicament container holder 9.

In FIG. 6, the medicament container holder 9 has been pushed further into the housing 3, i.e., in the proximal direction, by a proximally directed axial force greater than the threshold value. The medicament container holder 9 has reached its proximal position. The liquid container 13a has thus also moved in the proximal direction with the medicament container holder 9. The distally directed needle of the double-edged needle 13b has penetrated the septum as a result of the distal flexible tube being compressed and pierced by the distally directed needle.

The medicament container holder 9 is moved from the distal position to the proximal position by an interior structure, such as a motor frame, arranged in the drive unit when the cassette unit 1 is being connected to the drive unit.

FIG. 7 and FIG. 15 show the cassette unit 1 of the first embodiment when the lock member 7 is in the first position, and FIGS. 8-9 show the cassette unit when the lock member 7 is in the second position. In FIG. 9, the housing 3 has been removed to better illustrate the axial alignment of the axially extending rib 5d of the delivery member cover 7 and the axial passageway 7c of the lock member 7.

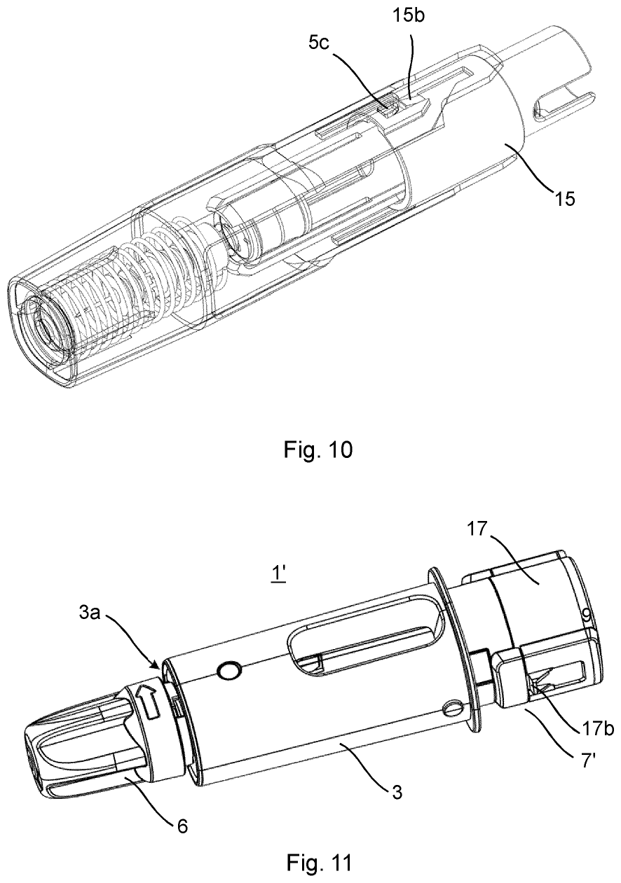

FIG. 10 shows the cassette unit 1 in a locked-out state when the delivery member cover 5 has been returned to the extended position. To illustrate the interaction between the rotator 15 and the delivery member cover 5, the delivery member cover 5 has been made transparent. One of the flexible elements 15c of the rotator 15 is shown arranged distally relative to the radially inwards extending protrusion 5c to prevent the delivery member cover 15 from moving back to the retracted position.

Furthermore, in another example, the cassette sub-assembly comprises a cap 6 removably attached to the proximal end 3b of the housing 3, as shown in FIGS. 11-12.

The cassette sub-assemblies and cassette units described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a medicament as listed herein (or a pharmaceutically acceptable salt of the medicament) and a pharmaceutically acceptable carrier. Pharmaceutical formulations comprising a drug as listed herein (or a pharmaceutically acceptable salt of the medicament) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are defined by the following clauses.
1. A cassette sub-assembly for a cassette unit (1) configured to be detachably connected to a drive unit to form a medicament delivery device, the cassette sub-assembly comprising:
   a housing (3) having a proximal housing end (3a) and a distal housing end (3b),
   a delivery member cover (5) protruding from the proximal housing end (3a) and configured to be moved linearly relative to the housing (3),
   a lock member (7) arranged axially fixed relative to the housing (3), the lock member (7) being configured to be rotated relative to the delivery member cover (5) from a first position to a second position,
   wherein the lock member (7) has a proximally facing radial surface (7b) and the delivery member cover (5) has a distally facing radial surface (5e),
   wherein the proximally facing radial surface (7b) is axially aligned with and facing the distally facing radial surface (5e) when the lock member (7) is in the first position and
   wherein the proximally facing radial surface (7b) is circumferentially offset relative to the distally facing radial surface (5e) when the lock member (7) is in the second position.
2. The cassette sub-assembly as described in clause 1, wherein the lock member (7) is arranged radially outside of the delivery member cover (5).
3. The cassette sub-assembly as described in clause 1 or 2, wherein the lock member (7) is annular, is a circlip or has a half-circular shape.
4. The cassette sub-assembly as described in any of the preceding clauses, wherein the delivery member cover (5) has an axially extending rib (5d), wherein a distal end face of the rib (5d) forms the distally facing radial surface (5e), and the lock member (7) has a ledge structure (7a) forming the proximally facing radial surface (7b) and a distally extending axial passageway (7c) next to the ledge structure (7a) in a circumferential direction, wherein the axial passageway (7c) is configured to receive the rib (5d) when the lock member (7) is in the second position.
5. The cassette sub-assembly as described in any of clauses 1-3, wherein the lock member (7) has an axially extending rib, wherein a proximal end face of the rib forms the proximally facing radial surface, and the delivery member cover (5) has a ledge structure forming the distally facing radial surface and a proximally extending axial passageway next to the ledge structure in a circumferential direction, wherein the axial passageway is configured to receive the rib when the lock member (7) is in the second position.
6. The cassette sub-assembly as described in clause 4 or 5, wherein the ledge structure (7a) comprises a radial ledge surface and a protrusion (7d) extending axially from the radial ledge surface, wherein the protrusion (7d) is arranged between the radial ledge surface and the axial passageway (7c) in the circumferential direction of the lock member (7).
7. The cassette sub-assembly as described in any of the preceding clauses, wherein the delivery member cover (5) is rotationally locked relative to the housing (3).
8. The cassette sub-assembly as described in clause 7, wherein the outer surface of the delivery member cover (5) has a perimeter that has a non-circular cross-sectional shape and the housing (3) has an inner surface with a cross-sectional shape that corresponds to the non-circular cross-sectional shape, the cross-sections being taken through a longitudinal axis of the cassette sub-assembly.
9. The cassette sub-assembly as described in clause 7, wherein the delivery member cover (5) has an outer surface provided with a first structure and the housing has a second structure, the first structure being configured to engage with the second structure to prevent relative rotation between the delivery member cover (5) and the housing (3).
10. The cassette sub-assembly as described in clause 9, wherein the first structure is an axial rib and the second structure is an axial channel, or wherein the first structure is an axial channel, and the second structure is an axial rib.
11. The cassette sub-assembly as described in any of the preceding clauses, comprising a fastening structure (17d) configured to engage with a counter fastening structure of the drive unit.
12. The cassette sub-assembly as described in clause 11, wherein the fastening structure (17c) forms one part of a bayonet connection or forms one part of a screw connection.
13. The cassette sub-assembly as described in any of the preceding clauses, comprising a medicament container holder (9) arranged in the housing (3) and configured to be moved from a distal position relative to the housing (3) to a proximal position by the drive unit when the cassette sub-assembly is connected to the drive unit.
14. The cassette sub-assembly as described in clause 13, wherein the medicament container holder (9) has an outer surface provided with a radially outwards extending bevelled surface (9a) and the housing (3) has an inner surface provided with a radially inwards extending bevelled surface (3e) arranged proximally relative to the radially outwards extending bevelled surface (9a) and facing the radially outwards extending bevelled surface (3e) when the medicament container holder (9) is in the distal position.
15. A cassette unit (1) for a medicament delivery device comprising the cassette sub-assembly as described in any of the preceding clauses.
16. A cassette sub-assembly for a cassette unit (1) configured to be detachably connected to a drive unit to form a medicament delivery device, the cassette sub-assembly comprising:
   a housing (3) having a proximal housing end (3a) and a distal housing end (3b),
   a delivery member cover (5) protruding from the proximal housing end (3a) and configured to be moved linearly relative to the housing (3),
   a lock member (7) arranged axially fixed relative to the housing (3), the lock member (7) being configured to be rotated relative to the delivery member cover (5) from a first position where an axial movement of the delivery member cover is blocked by the lock member (7) to a second position where the axial movement of the delivery member cover is no longer blocked by the lock member (7),
   a rear member (17) arranged distally relative to the lock member (7); wherein the rear member (17) provided with a radially flexible structure (17b) with an axially extending end tab (17c); wherein the axially extending end tab (17c) is received by a respective axial recess (7g) of the lock member (7) when the lock member (7) is in the first position such that the lock member (7) is rotationally fixed in the first position to the rear member (17); and
   wherein the radially flexible structure (17b) is configured to be pushed radially inwards by an inner wall of the drive unit when the cassette unit (1) is inserted into and connected to the drive unit such that the radially flexible structure (17b) is disengaged from the axial recess (7g) of the lock member (7).
17. The cassette sub-assembly as described in clause 16, wherein the lock member (7) is arranged radially outside of the delivery member cover (5).
18. The cassette sub-assembly as described in clause 16 or 17, wherein the lock member (7) is annular, is a circlip or has a half-circular shape.
19. The cassette sub-assembly as described in any clauses 16-18, wherein the delivery member cover (5) is rotationally locked relative to the housing (3).
20. The cassette sub-assembly as described in clause 19, wherein the outer surface of the delivery member cover (5) has a perimeter that has a non-circular cross-sectional shape and the housing (3) has an inner surface with a cross-sectional shape that corresponds to the non-circular cross-sectional shape, the cross-sections being taken through a longitudinal axis of the cassette sub-assembly.
21. The cassette sub-assembly as described in any of clauses 16-20, wherein the rear member (17) comprises a fastening structure (17d) configured to engage with a counter fastening structure of the drive unit.
22. The cassette sub-assembly as described in clause 21, wherein the fastening structure (17c) forms one part of a bayonet connection or forms one part of a screw connection.
23. The cassette sub-assembly as described in any of clauses 16-22, wherein the rear member (17) has a proximal rear member end (17a) provided with the radially flexible structure (17b).
24. The cassette sub-assembly as described in clause 23, wherein the radially flexible structure (17b) is wedge-shaped, with an increasing radial dimension in a direction towards the proximal rear member end (17a).
25. The cassette sub-assembly as described in any of clauses 16-24, wherein the lock member (7) has a proximally facing radial surface (7b); wherein the delivery member cover (5) has a distally facing radial surface (5e); wherein the proximally facing radial surface (7b) is axially aligned with and facing the distally facing radial surface (5e) when the lock member (7) is in the first position; and
   wherein the proximally facing radial surface (7b) is circumferentially offset relative to the distally facing radial surface (5e) when the lock member (7) is in the second position.
26. The cassette sub-assembly as described in any of clauses 16-24, wherein the lock member has a distally facing radial surface; wherein the delivery member cover has a proximally facing radial surface; wherein the proximally facing radial surface is axially aligned with and facing the distally facing radial surface when the lock member is in the first position; and wherein the proximally facing radial surface is circumferentially offset relative to the distally facing radial surface when the lock member is in the second position.
27. The cassette sub-assembly as described in clause 26, wherein the distally facing radial surface of the lock member is a helical surface.
28. The cassette sub-assembly as described in any of clauses 16-27, wherein the cassette sub-assembly comprises a resilient member (11); and wherein the resilient member (11) is configured to bias the delivery member cover (5) towards the extended position.
29. A cassette unit (1) for a medicament delivery device comprising the cassette sub-assembly as described in any of clauses 16-28.
30. A medicament delivery device comprises a cassette unit (1) according to clause 29, and a drive unit configured to be connected to the cassette unit (1).

1-15. (canceled)

16. A cassette sub-assembly for a cassette unit configured to be detachably connected to a drive unit to form a medicament delivery device, the cassette sub-assembly comprising:
a housing having a proximal housing end and a distal housing end;
a delivery member cover protruding from the proximal housing end (3a) and configured to be moved linearly relative to the housing;
a lock member arranged axially fixed relative to the housing, the lock member being configured to be rotated relative to the delivery member cover from a first position where an axial movement of the delivery member cover is blocked by the lock member to a second position where the axial movement of the delivery member cover is no longer blocked by the lock member; and
a rear member arranged distally relative to the lock member, wherein the rear member provided with a radially flexible structure with an axially extending end tab, and wherein the axially extending end tab is received by a respective axial recess of the lock member when the lock member is in the first position such that the lock member is rotationally fixed in the first position to the rear member,
wherein the radially flexible structure is configured to be pushed radially inwards by an inner wall of the drive unit when the cassette unit is inserted into and connected to the drive unit such that the radially flexible structure is disengaged from the axial recess of the lock member.

17. The cassette sub-assembly of claim 16, wherein the lock member is arranged radially outside of the delivery member cover.

18. The cassette sub-assembly of claim 16, wherein the lock member is annular or has a half-circular shape.

19. The cassette sub-assembly of claim 16, wherein the delivery member cover is rotationally locked relative to the housing.

20. The cassette sub-assembly of claim 19, wherein the outer surface of the delivery member cover has a perimeter that has a non-circular cross-sectional shape and the housing has an inner surface with a cross-sectional shape that corresponds to the non-circular cross-sectional shape, the cross-sections being taken through a longitudinal axis of the cassette sub-assembly.

21. The cassette sub-assembly of claim 16, wherein the rear member comprises a fastening structure configured to engage with a counter fastening structure of the drive unit.

22. The cassette sub-assembly of claim 21, wherein the fastening structure forms one part of a bayonet connection or forms one part of a screw connection.

23. The cassette sub-assembly of claim 16, wherein the rear member has a proximal rear member end provided with the radially flexible structure.

24. The cassette sub-assembly of claim 23, wherein the radially flexible structure is wedge-shaped, with an increasing radial dimension in a direction towards the proximal rear member end.

25. The cassette sub-assembly of claim 16, wherein the lock member has a proximally facing radial surface, wherein the delivery member cover has a distally facing radial surface, wherein the proximally facing radial surface is axially aligned with and facing the distally facing radial surface when the lock member is in the first position, and wherein the proximally facing radial surface is circumferentially offset relative to the distally facing radial surface when the lock member is in the second position.

26. The cassette sub-assembly of claim 16, wherein the lock member has a distally facing radial surface; wherein the delivery member cover has a proximally facing radial surface, wherein the proximally facing radial surface is axially aligned with and facing the distally facing radial surface when the lock member is in the first position, and wherein the proximally facing radial surface is circumferentially offset relative to the distally facing radial surface when the lock member is in the second position.

27. The cassette sub-assembly of claim 26, wherein the distally facing radial surface of the lock member is a helical surface.

28. The cassette sub-assembly of claim 16, wherein the cassette sub-assembly comprises a resilient member, and wherein the resilient member is configured to bias the delivery member cover towards the extended position.

29. A cassette unit for a medicament delivery device comprising the cassette sub-assembly of claim 16.

30. A medicament delivery device comprising:
the cassette unit according to claim 29; and
a drive unit configured to be connected to the cassette unit.
